(19) United States
(12) Patent Application Publication
(10) Pub. No.: US 2021/0221817 A1
(43) Pub. Date: Jul. 22, 2021

(54) NOVEL HETEROAROMATIC COMPOUNDS AS POTENT MODULATORS OF THE HIPPO-YAP SIGNALING PATHWAY LATS1/2 KINASES

(71) Applicant: Nivien Therapeutics Compny, Allston, MA (US)

(72) Inventors: Nathaniel B. Horwitz, West Tisbury, MA (US); Nikita Shah, Lithia, FL (US); Wenkui Ken Fang, Irvine, CA (US); Dhrmendra Singh, Houston, TX (US)

(21) Appl. No.: 17/270,980

(22) PCT Filed: Aug. 28, 2019
(86) PCT No.: PCT/US19/48474

§ 371 Date: Feb. 24, 2021

Related U.S. Application Data
(60) Provisional application No. 62/725,463, filed on Aug. 31, 2018.

(51) Int. Cl. C07D 487/04; C07D 471/04; A61K 45/06

(52) U.S. Cl. CPC C07D 487/04; A61K 45/06; C07D 471/04

(57) ABSTRACT

The present invention relates to novel heteroaromatic compounds of formulae I and II and pharmaceutically acceptable salts thereof. The compounds of the invention are useful as inhibitors of serine/threonine protein kinases LATS1 and LATS2. Also provided herein are processes for making compounds of formulae I and II, and pharmaceutical compositions comprising the compounds of the invention. The present invention also provides methods of treating cancer with a compound of formula I or II.

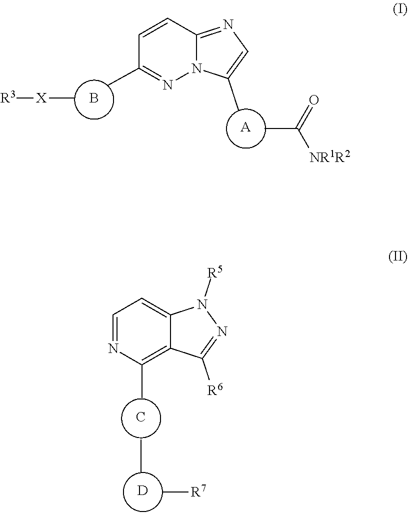

### RELATED APPLICATION

This application claims the benefit of priority to U.S. Provisional Patent Application No. 62/725,463, filed Aug. 31, 2018, which application is hereby incorporated by reference in its entirety.

### BACKGROUND OF THE INVENTION

LATS1 and LATS2 are a pair of highly-homologous serine/threonine protein kinases that function as enzymes at the core of the Hippo-YAP signaling pathway. When the Hippo pathway is active—or “ON”—a series of upstream factors phosphorylate the Hippo kinases MST1/2, which in turn phosphorylate LATS1/2. LATS1/2 phosphorylates Yes-associated protein (YAP), causing its cytoplasmic retention and ubiquitin-mediated degradation. When the Hippo pathway is inactive—or “OFF”—and LATS1/2 is eliminated, reduced, and/or not phosphorylated, YAP is not phosphorylated and instead localizes to the nucleus. In the nucleus, YAP complexes with the TEAD family of transcription factors to regulate a series of downstream genes relevant to functions including cancer resistance, growth regulation and other cellular properties. Therefore, inhibition of LATS1/2 is an effective method to inactivate the Hippo pathway and cause de-phosphorylation and nuclear localization of YAP.

When YAP is localized to the nucleus, there are two potentially therapeutic downstream effects: First, cancer cell lines that were previously resistant to cytotoxic drugs become sensitized to the cytotoxic drugs—both in vitro and in vivo (Gujral, Taran; Kirschner, Marc, 2016, PNAS). Second, an immune cascade is triggered that results in increased proliferation of cytotoxic CD8+ T-cells (Moroishi, et al., 2017, Cell).

In cytotoxic sensitization, Hippo inactivation reduces promoter and transcription levels of drug-metabolizing enzymes such as cytidine deaminase (CDA) and a suite of multidrug efflux transporters (ABCG2, ABCC3, MVP/LRP, and several others). Reductions in drug-metabolizing and drug-export factors increase intracellular levels of cytotoxic drugs, including imatinib, gemcitabine, 5-fluorouracil, and small molecules from several other classes of therapeutic agents. Increased intracellular levels of cytotoxic drugs result in increased cell death of previously drug-resistant patient-derived cancer cell lines, in cancer types including pancreatic ductal adenocarcinoma (PDAC), ovarian cancer, hepatocellular carcinoma (HCC), glioblastoma mellitus (GBM), and others, as well as in patient-derived xenograft (PDX) mouse models of PDAC. Naturally-occurring mutations in the Hippo pathway also correlate with improved median overall survival (mOS) in retrospective cohorts of patients with PDAC treated with gemcitabine. Thus, inactivating the Hippo pathway via inhibition of LATS1/2 has the potential to sensitize drug-resistant cancers to treatment with a range of cytotoxic drugs.

In immune enhancement, Hippo inactivation stimulates secretion of nucleic acid-rich extracellular vesicles (EVs) that trigger a type 1 interferon gamma response via Toll-like receptors 7 and 9 (TLR7/9) and the MYD88-TRIF pathway. This immune response leads to increased CD8+ T-cell infiltration in syngeneic mouse models of melanoma, breast cancer, and head and neck cancer, and promotes immune memory. Therefore, inactivating the Hippo pathway via inhibition of LATS1/2 may trigger increased CD8+ T-cell infiltration in previously non-immunogenic tumor types and promote immune memory.

Accordingly, there exists a need to develop inhibitors of LATS1/2 kinases and use such inhibitors in a therapeutic regimen for treating cancer.

### SUMMARY OF THE INVENTION

In some aspects, the invention provides a compound of formula I,

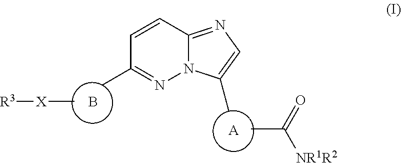

or a pharmaceutically acceptable salt thereof;
wherein:
ring

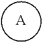

represents substituted or unsubstituted arylene or heteroarylene;
ring

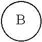

represents substituted or unsubstituted arylene, heteroarylene, heterocycloalkylene, heterocycloalkenylene, cycloalkylene, or cycloalkenylene;
R¹ and R² each independently represent H, alkyl, or cycloalkyl;
X represents a bond, O, S, or NH; and
R³ represents H, alkyl, cycloalkyl, (cycloalkyl)alkyl, heterocycloalkyl, (heterocycloalkyl)alkyl, aryl, arylalkyl, heteroaryl, or heteroarylalkyl.

In further aspects, the invention provides a compound of formula II,

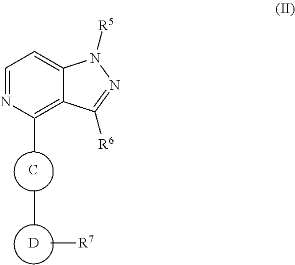

or a pharmaceutically acceptable salt thereof;
wherein:
ring

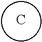

represents substituted or unsubstituted arylene or heteroarylene;
ring

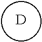

represents substituted or unsubstituted heterocycloalkylene or heterocycloalkenylene;
R⁵ represents H or alkyl;
R⁶ represents H, alkyl, alkoxy, or amino; and
R⁷ represents substituted or unsubstituted aryl or heteroaryl.

Also provided herein are pharmaceutical compositions comprising a compound of the invention and a pharmaceutically acceptable carrier.

Further provided are methods of treating cancer with a compound of the invention.

### BRIEF DESCRIPTION OF THE DRAWINGS

FIG. 1 is an image showing that the LATS1/2 inhibitors of the invention reduce YAP phosphorylation in PDAC PANC1 cells in a dose-dependent manner. FIG. 1 consists of two columns. On the left: the cells were treated with control and 5 μM of each of the compounds of the invention. Control in lane “C.” In lane “1” is 4-(6-(4-(2-morpholinoethoxy)phenyl)imidazo[1,2-b]pyridazin-3- yl)benzamide (compound 1). In lane “2” is 4-(6-(3-aminopiperidin-1-yl)imidazo[1,2-b]pyridazin-3-yl)benzamide (compound 3). In lane “4” is 4-(6-(4-aminopiperidin-1-yl)imidazo[1,2-b]pyridazin-3-yl)benzamide (compound 4). In lane “6” is 4-(6-(piperazin-1-yl)imidazo[1,2-b]pyridazin-3-yl)benzamide (compound 2). In lane “7” is 4-(4-(4-(4-chlorophenyl)piperidin-4-yl)phenyl)-3-methyl-1H-pyrazolo[4,3-c]pyridine (compound 5).

On the left: the cells were treated with control and 25 μM of each of the compounds of the invention. Control in lane “C.” In lane “1” is 4-(6-(4-(2-morpholinoethoxy)phenyl)imidazo[1,2-b]pyridazin-3-yl)benzamide (compound 1). In lane “2” is 4-(6-(3-aminopiperidin-1-yl)imidazo[1,2-b]pyridazin-3-yl)benzamide (compound 3). In lane “4” is 4-(6-(4-aminopiperidin-1-yl)imidazo[1,2-b]pyridazin-3-yl)benzamide (compound 4). In lane “6” is 4-(6-(piperazin-1-yl)imidazo[1,2-b]pyridazin-3-yl)benzamide (compound 2). In lane “7” is 4-(4-(4-(4-chlorophenyl)piperidin-4-yl)phenyl)-3-methyl-1H-pyrazolo[4,3-c]pyridine (Compound 5). Compound 5, 4-(4-(4-(4-chlorophenyl)piperidin-4-yl)phenyl)-3-methyl-1H-pyrazolo[4,3-c]pyridine, killed all PANC1 cells at this dosage.

FIG. 2 is an image showing that the LATS1/2 inhibitors of the invention reduce transcription of ABCG2, a critical multidrug efflux transporter, in PDAC PANC1 cells. The y-axis is the normalized transcription level of ABCG2. The bar labeled ABCG2_control represents PANC1 cells that received no treatment. The bar labeled ABCG2_201 represents PANC1 cells treated with 4-(6-(4-(2-morpholinoethoxy)phenyl)imidazo[1,2-b]pyridazin-3-yl)benzamide (compound 1) at 25 μM. The bar labeled ABCG2_206 represents cells treated with 4-(6-(piperazin-1-yl)imidazo[1,2-b]pyridazin-3-yl)benzamide (compound 2) 25 μM.

### DETAILED DESCRIPTION OF THE INVENTION

The present invention relates to heteroaromatic compounds useful as potent inhibitors of LATS1/2 kinases. Inhibition of these kinases, which are part of the Hippo-YAP signaling pathway, can stimulate an anti-cancer immune response and reduce cytotoxic drug resistance in cancer patients. Accordingly, the invention also relates to the use of these LATS1/2 inhibitor compounds for treating disorders associated with LATS1/2 modulation, such as cancer.

### Definitions

The articles “a” and “an” are used herein to refer to one or to more than one (i.e., to at least one) of the grammatical object of the article. By way of example, “an element” means one element or more than one element.

The term “heteroatom” is art-recognized and refers to an atom of any element other than carbon or hydrogen. Illustrative heteroatoms include boron, nitrogen, oxygen, phosphorus, sulfur and selenium, and alternatively oxygen, nitrogen or sulfur.

The term “alkyl” as used herein is a term of art and refers to saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, the term “alkyl” as refers to saturated aliphatic groups, including straight-chain alkyl groups and branched-chain alkyl groups. In certain embodiments, a straight-chain or branched-chain alkyl has about 30 or fewer carbon atoms in its backbone (e.g., C₁-C₃₀ for straight chain, C₃-C₃₀ for branched chain), and alternatively, about 20 or fewer, or 10 or fewer. In certain embodiments, the term “alkyl” refers to a C₁-C₁₀ alkyl group. In certain embodiments, the term “alkyl” refers to a C₁-C₆ alkyl group, for example a C₁-C₆ straight-chain alkyl group. In certain embodiments, the term “alkyl” refers to a C₃-C₁₂ branched-chain alkyl group. In certain embodiments, the term “alkyl” refers to a C₃-C₈ branched-chain alkyl group. Representative examples of alkyl include, but are not limited to, methyl, ethyl, n-propyl, iso-propyl, n-butyl, sec-butyl, iso-butyl, tert-butyl, n-pentyl, isopentyl, neopentyl, and n-hexyl.

The term “cycloalkyl” means mono- or bicyclic or bridged saturated carbocyclic rings, each having from 3 to 12 carbon atoms. Certain cycloalkyls have from 5-12 carbon atoms in their ring structure, and may have 6-10 carbons in the ring structure. Preferably, cycloalkyl is (C₃-C₇)cycloalkyl, which represents a monocyclic saturated carbocyclic ring, having from 3 to 7 carbon atoms. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems include bridged monocyclic rings and fused bicyclic rings. Bridged monocyclic rings contain a monocyclic cycloalkyl ring where two non-adjacent carbon atoms of the monocyclic ring are linked by an alkylene bridge of between one and three additional carbon atoms (i.e., a bridging group of the form —(CH₂)_{w}—, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. The bridged or fused bicyclic cycloalkyl is attached to the parent molecular moiety through any carbon atom contained within the monocyclic cycloalkyl ring. Cycloalkyl groups are optionally substituted. In certain embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted.

The term “cycloalkenyl”, as used herein, refers to mono- or bicyclic or bridged carbocyclic rings having 3 to 12 carbon atoms, more preferably 5 to 8 carbon atoms, having at least one carbon-carbon double bond within the ring.

The term “(cycloalkyl)alkyl” as used herein refers to an alkyl group substituted with one or more cycloalkyl groups. An example of cycloalkylalkyl is cyclohexylmethyl group.

The term “heterocycloalkyl” or “heterocyclyl” as used herein refers to a radical of a non-aromatic ring system, including, but not limited to, monocyclic, bicyclic, and tricyclic rings, which can be completely saturated or which can contain one or more units of unsaturation, for the avoidance of doubt, the degree of unsaturation does not result in an aromatic ring system, and having 3 to 12 atoms including at least one heteroatom, such as nitrogen, oxygen, or sulfur. For purposes of exemplification, which should not be construed as limiting the scope of this invention, the following are examples of heterocyclic rings: aziridinyl, azirinyl, oxiranyl, thiiranyl, thiirenyl, dioxiranyl, diazirinyl, diazepanyl, 1,3-dioxanyl, 1,3-dioxolanyl, 1,3-dithiolanyl, 1,3-dithianyl, imidazolidinyl, isothiazolinyl, isothiazolidinyl, isoxazolinyl, isoxazolidinyl, azetyl, oxetanyl, oxetyl, thietanyl, thietyl, diazetidinyl, dioxetanyl, dioxetenyl, dithietanyl, dithietyl, dioxalanyl, oxazolyl, thiazolyl, triazinyl, isothiazolyl, isoxazolyl, azepines, azetidinyl, morpholinyl, oxadiazolinyl, oxadiazolidinyl, oxazolinyl, oxazolidinyl, oxopiperidinyl, oxopyrrolidinyl, piperazinyl, piperidinyl, pyranyl, pyrazolinyl, pyrazolidinyl, pyrrolinyl, pyrrolidinyl, quinuclidinyl, thiomorpholinyl, tetrahydropyranyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. A heterocycloalkyl group is optionally substituted by one or more substituents as described below.

The term “(heterocycloalkyl)alkyl” as used herein refers to an alkyl group substituted with one or more heterocycloalkyl (i.e., heterocyclyl) groups.

The term “alkenyl” as used herein means a straight or branched chain hydrocarbon radical containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl. The unsaturated bond(s) of the alkenyl group can be located anywhere in the moiety and can have either the (Z) or the (E) configuration about the double bond(s).

The term “alkynyl” as used herein means a straight or branched chain hydrocarbon radical containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “alkylene” is art-recognized, and as used herein pertains to a diradical obtained by removing two hydrogen atoms of an alkyl group, as defined above. In one embodiment an alkylene refers to a disubstituted alkane, i.e., an alkane substituted at two positions with substituents such as halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluromethyl), cyano, or the like. That is, in one embodiment, a “substituted alkyl” is an “alkylene”.

The term “amino” is a term of art and as used herein refers to both unsubstituted and substituted amines, e.g., a moiety that may be represented by the general formulas:

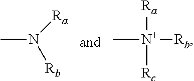

wherein Rₐ, R_{b}, and R_{c} each independently represent a hydrogen, an alkyl, an alkenyl, —(CH₂)ₓ—R_{d}, or Rₐ and R_{b}, taken together with the N atom to which they are attached complete a heterocycle having from 4 to 8 atoms in the ring structure; R_{d} represents an aryl, a cycloalkyl, a cycloalkenyl, a heterocyclyl or a polycyclyl; and x is zero or an integer in the range of 1 to 8. In certain embodiments, only one of Rₐ or R_{b} may be a carbonyl, e.g., Rₐ, R_{b}, and the nitrogen together do not form an imide. In other embodiments, Rₐ and R_{b} (and optionally R_{c}) each independently represent a hydrogen, an alkyl, an alkenyl, or —(CH₂)ₓ—R_{d}. In certain embodiments, the term “amino” refers to —NH₂.

In certain embodiments, the term “alkylamino” refers to —NH(alkyl).

In certain embodiments, the term “dialkylamino” refers to —N(alkyl)₂.

The term “amido”, as used herein, means —NHC(═O)—, wherein the amido group is bound to the parent molecular moiety through the nitrogen. Examples of amido include alkylamido such as CH₃C(═O)N(H)— and CH₃CH₂C(═O)N(H)—. The term “amide” also encompasses a group of formula “—C(O)NR^{x}R^{y},” wherein R^{x} and R^{y} can be the same or different, and each of R^{x} and R^{y} are independently selected from H, alkyl, aralkyl, aryl, heteroarylalkyl, heteroaryl, cycloalkyl, cycloalkenyl, and heterocyclyl.

The term “acyl” is a term of art and as used herein refers to any group or radical of the form RCO— where R is any organic group, e.g., alkyl, aryl, heteroaryl, aralkyl, and heteroaralkyl. Representative acyl groups include acetyl, benzoyl, and malonyl.

The term “aminoalkyl” as used herein refers to an alkyl group substituted with one or more one amino groups. In one embodiment, the term “aminoalkyl” refers to an aminomethyl group.

The term “aminoacyl” is a term of art and as used herein refers to an acyl group substituted with one or more amino groups.

The term “aminothionyl” as used herein refers to an analog of an aminoacyl in which the O of RC(O)— has been replaced by sulfur, hence is of the form RC(S)—.

The term “phosphoryl” is a term of art and as used herein may in general be represented by the formula:

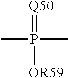

wherein Q50 represents S or O, and R59 represents hydrogen, a lower alkyl or an aryl; for example, —P(O)(OMe)- or —P(O)(OH)₂. When used to substitute, e.g., an alkyl, the phosphoryl group of the phosphorylalkyl may be represented by the general formulas:

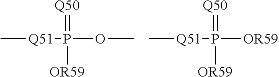

wherein Q50 and R59, each independently, are defined above, and Q51 represents O, S or N; for example, —O—P(O)(OH)OMe or —NH—P(O)(OH)₂. When Q50 is S, the phosphoryl moiety is a “phosphorothioate.”

The term “aminophosphoryl” as used herein refers to a phosphoryl group substituted with at least one amino group, as defined herein; for example, —P(O)(OH)NMe₂.

The term “azide” or “azido,” as used herein, means an —N₃ group.

The term “carbonyl” as used herein refers to —C(═O)—.

The term “thiocarbonyl” as used herein refers to —C(═S)—.

The term “alkylphosphoryl” as used herein refers to a phosphoryl group substituted with at least one alkyl group, as defined herein; for example, —P(O)(OH)Me.

The term “alkylthio” as used herein refers to alkyl-S—. The term “(alkylthio)alkyl” refers to an alkyl group substituted by an alkylthio group.

The term “carboxy,” as used herein, means a —CO₂H group.

The term “aryl” is a term of art and as used herein refers to includes monocyclic, bicyclic and polycyclic aromatic hydrocarbon groups, for example, benzene, naphthalene, anthracene, and pyrene. Typically, an aryl group contains from 6-10 carbon ring atoms (i.e., (C₆-C₁₀)aryl). The aromatic ring may be substituted at one or more ring positions with one or more substituents, such as halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluromethyl), cyano, or the like. The term “aryl” also includes polycyclic ring systems having two or more cyclic rings in which two or more carbons are common to two adjoining rings (the rings are “fused rings”) wherein at least one of the rings is an aromatic hydrocarbon, e.g., the other cyclic rings may be cycloalkyls, cycloalkenyls, cycloalkynyls, aryls, heteroaryls, and/or heterocyclyls. In certain embodiments, the term “aryl” refers to a phenyl group.

The term “heteroaryl” is a term of art and as used herein refers to a monocyclic, bicyclic, and polycyclic aromatic group having 3 to 12 total atoms including one or more heteroatoms such as nitrogen, oxygen, or sulfur in the ring structure. Exemplary heteroaryl groups include azaindolyl, benzo(b)thienyl, benzimidazolyl, benzofuranyl, benzoxazolyl, benzothiazolyl, benzothiadiazolyl, benzotriazolyl, benzoxadiazolyl, furanyl, imidazolyl, imidazopyridinyl, indolyl, indolinyl, indazolyl, isoindolinyl, isoxazolyl, isothiazolyl, isoquinolinyl, oxadiazolyl, oxazolyl, purinyl, pyranyl, pyrazinyl, pyrazolyl, pyridinyl, pyrimidinyl, pyrrolyl, pyrrolo[2,3-d]pyrimidinyl, pyrazolo[3,4-d]pyrimidinyl, quinolinyl, quinazolinyl, triazolyl, thiazolyl, thiophenyl, tetrahydroindolyl, tetrazolyl, thiadiazolyl, thienyl, thiomorpholinyl, triazolyl or tropanyl, and the like. The “heteroaryl” may be substituted at one or more ring positions with one or more substituents such as halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluromethyl), cyano, or the like. The term “heteroaryl” also includes polycyclic ring systems having two or more cyclic rings in which two or more carbons are common to two adjoining rings (the rings are “fused rings”) wherein at least one of the rings is an aromatic group having one or more heteroatoms in the ring structure, e.g., the other cyclic rings may be cycloalkyls, cycloalkenyls, cycloalkynyls, aryls, heteroaryls, and/or heterocyclyls.

The term “aralkyl” or “arylalkyl” is a term of art and as used herein refers to an alkyl group substituted with an aryl group, wherein the moiety is appended to the parent molecule through the alkyl group.

The term “heteroaralkyl” or “heteroarylalkyl” is a term of art and as used herein refers to an alkyl group substituted with a heteroaryl group, appended to the parent molecular moiety through the alkyl group.

The term “alkoxy” as used herein means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxyalkyl” refers to an alkyl group substituted by an alkoxy group.

The term “alkoxycarbonyl” means an alkoxy group, as defined herein, appended to the parent molecular moiety through a carbonyl group, represented by —C(═O)—, as defined herein. Representative examples of alkoxycarbonyl include, but are not limited to, methoxycarbonyl, ethoxycarbonyl, and tert-butoxycarbonyl.

The term “alkylcarbonyl,” as used herein, means an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “arylcarbonyl,” as used herein, means an aryl group, as defined herein, appended to the parent molecular moiety through a carbonyl group, as defined herein. Representative examples of arylcarbonyl include, but are not limited to, benzoyl and (2-pyridinyl)carbonyl.

The term “alkylcarbonyloxy” and “arylcarbonyloxy,” as used herein, means an alkylcarbonyl or arylcarbonyl group, as defined herein, appended to the parent molecular moiety through an oxygen atom. Representative examples of alkylcarbonyloxy include, but are not limited to, acetyloxy, ethylcarbonyloxy, and tert-butylcarbonyloxy. Representative examples of arylcarbonyloxy include, but are not limited to phenylcarbonyloxy.

The term “alkenoxy” or “alkenoxyl” means an alkenyl group, as defined herein, appended to the parent molecular moiety through an oxygen atom. Representative examples of alkenoxyl include, but are not limited to, 2-propen-1-oxyl (i.e., CH₂═CH—CH₂—O—) and vinyloxy (i.e., CH₂═CH—O—).

The term “aryloxy” as used herein means an aryl group, as defined herein, appended to the parent molecular moiety through an oxygen atom.

The term “heteroaryloxy” as used herein means a heteroaryl group, as defined herein, appended to the parent molecular moiety through an oxygen atom.

The term “carbocyclyl” as used herein means a monocyclic or multicyclic (e.g., bicyclic, tricyclic, etc.) hydrocarbon radical containing from 3 to 12 carbon atoms that is completely saturated or has one or more unsaturated bonds, and for the avoidance of doubt, the degree of unsaturation does not result in an aromatic ring system (e.g., phenyl). Examples of carbocyclyl groups include 1-cyclopropyl, 1-cyclobutyl, 2-cyclopentyl, 1-cyclopentenyl, 3-cyclohexyl, 1-cyclohexenyl and 2-cyclopentenylmethyl.

The term “cyano” is a term of art and as used herein refers to —CN.

The term “halo” is a term of art and as used herein refers to —F, —Cl, —Br, or —I.

The term “nitro” as used herein refers to a group of formula “—NO₂”.

The term “haloalkyl” as used herein refers to an alkyl group, as defined herein, wherein some or all of the hydrogens are replaced with halogen atoms.

The term “hydroxy” is a term of art and as used herein refers to —OH.

The term “hydroxyalkyl”, as used herein, means at least one hydroxy group, as defined herein, is appended to the parent molecular moiety through an alkyl group, as defined herein. Representative examples of hydroxyalkyl include, but are not limited to, hydroxymethyl, 2-hydroxyethyl, 3-hydroxypropyl, 2,3-dihydroxypentyl, and 2-ethyl-4-hydroxyheptyl.

The term “silyl”, as used herein, includes hydrocarbyl derivatives of the silyl (H₃Si—) group (i.e., (hydrocarbyl)₃Si—), wherein a hydrocarbyl groups are univalent groups formed by removing a hydrogen atom from a hydrocarbon, e.g., ethyl, phenyl. The hydrocarbyl groups can be combinations of differing groups which can be varied in order to provide a number of silyl groups, such as trimethylsilyl (TMS), tert-butyldiphenylsilyl (TBDPS), tert-butyldimethylsilyl (TBS/TBDMS), triisopropylsilyl (TIPS), and [2-(trimethylsilyl)ethoxy]methyl (SEM).

The term “silyloxy,” as used herein, means a silyl group, as defined herein, is appended to the parent molecule through an oxygen atom.

Certain compounds contained in compositions of the present invention may exist in particular geometric or stereoisomeric forms. In addition, compounds of the present invention may also be optically active. The present invention contemplates all such compounds, including cis- and trans-isomers, (R)- and (S)-enantiomers, diastereoisomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

As will be evident to those skilled in the art, individual isomeric forms can be obtained by separation of mixtures thereof in conventional manner. For example, in the case of diasteroisomeric isomers, chromatographic separation may be employed.

The present invention also includes all pharmaceutically acceptable isotopically enriched compounds. Any compound of the invention may contain one or more isotopic atoms enriched or different than the natural ratio such as deuterium ²H (or D) in place of protium ¹H (or H) or use of ¹³C enriched material in place of ¹²C and the like. Similar substitutions can be employed for N, O and S. The use of isotopes may assist in analytical as well as therapeutic aspects of the invention. For example, use of deuterium may increase the in vivo half-life by altering the metabolism (rate) of the compounds of the invention. These compounds can be prepared in accord with the preparations described by use of isotopically enriched reagents.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, fragmentation, decomposition, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

For example, in embodiments in which a substituent is substituted with one or more substituents, such substituents may be selected from substituted or unsubstituted alkyl, such as perfluoroalkyl (e.g., trifluoromethyl), alkenyl, alkoxy, alkoxyalkyl, aryl, aralkyl, arylalkoxy, aryloxy, aryloxyalkyl, hydroxyl, halo, alkoxy, such as perfluoroalkoxy (e.g., trifluoromethylalkoxy), alkoxyalkoxy, hydroxyalkyl, hydroxyalkylamino, hydroxyalkoxy, amino, aminoalkyl, alkylamino, aminoalkylalkoxy, aminoalkoxy, acylamino, acylaminoalkyl, such as perfluoro acylaminoalkyl (e.g., trifluoromethylacylaminoalkyl), acyloxy, cycloalkyl, cycloalkylalkyl, cycloalkylalkoxy, heterocyclyl, heterocyclylalkyl, heterocyclyloxy, heterocyclylalkoxy, heteroaryl, heteroarylalkyl, heteroarylalkoxy, heteroaryloxy, heteroaryloxyalkyl, heterocyclylaminoalkyl, heterocyclylaminoalkoxy, amido, amidoalkyl, amidine, imine, oxo, carbonyl (such as carboxyl, alkoxycarbonyl, formyl, or acyl, including perfluoroacyl (e.g., C(O)CF₃)), carbonylalkyl (such as carboxyalkyl, alkoxycarbonylalkyl, formylalkyl, or acylalkyl, including perfluoroacylalkyl (e.g., -alkylC(O)CF₃)), carbamate, carbamatealkyl, urea, ureaalkyl, sulfate, sulfonate, sulfamoyl, sulfone, sulfonamide, sulfonamidealkyl, cyano, nitro, azido, sulfhydryl, alkylthio, thiocarbonyl (such as thioester, thioacetate, or thioformate), phosphoryl, phosphate, phosphonate or phosphinate.

The phrase “protecting group,” as used herein, means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991). Protected forms of the inventive compounds are included within the scope of this invention.

For purposes of the invention, the chemical elements are identified in accordance with the Periodic Table of the Elements, CAS version, Handbook of Chemistry and Physics, 67th Ed., 1986-87, inside cover.

Other chemistry terms herein are used according to conventional usage in the art, as exemplified by The McGraw-Hill Dictionary of Chemical Terms (ed. Parker, S., 1985), McGraw-Hill, San Francisco, incorporated herein by reference). Unless otherwise defined, all technical and scientific terms used herein have the same meaning as commonly understood by one of ordinary skill in the art to which this invention pertains

The term “ketone” as used herein, represents an organic compound having a carbonyl group linked to a carbon atom such as —(CO)R^{x} wherein R^{x} can be alkyl, aralkyl, aryl, heteroarylalkyl, heteroaryl, cycloalkyl, cycloalkenyl, and heterocyclyl.

The term “aldehyde” as used herein, represents a group of formula “—C(O)H”.

The term “ester” as used herein, represents a group of formula “—C(O)OR^{x}”, wherein R^{x} can be H, alkyl, aralkyl, aryl, heteroarylalkyl, heteroaryl, cycloalkyl, cycloalkenyl, and heterocyclyl as defined above.

The term “sulfonamide” as used herein, represents a group of formula “—S(O)₂NR^{x}R^{y}” wherein R^{x} and R^{y} can be the same or independently H, alkyl, aralkyl, aryl, heteroarylalkyl, heteroaryl, cycloalkyl, cycloalkenyl, and heterocyclyl.

The term “sulfoxide” as used herein, represents a group of formula “—S═O”.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “prodrug” is intended to encompass compounds which, under physiologic conditions, are converted into the therapeutically active agents of the present invention (e.g., a compound of formula I or II). A common method for making a prodrug is to include one or more selected moieties which are hydrolyzed under physiologic conditions to reveal the desired molecule. In other embodiments, the prodrug is converted by an enzymatic activity of the host animal. For example, esters or carbonates (e.g., esters or carbonates of alcohols or carboxylic acids) are preferred prodrugs of the present invention. In certain embodiments, some or all of the compounds of formula I in a formulation represented above can be replaced with the corresponding suitable prodrug, e.g., wherein a hydroxyl in the parent compound is presented as an ester or a carbonate or carboxylic acid present in the parent compound is presented as an ester.

### Compounds of the Invention

The present invention provides a compound of formula I,

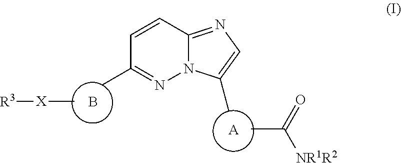

or a pharmaceutically acceptable salt thereof;
wherein:
ring

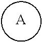

represents substituted or unsubstituted arylene or heteroarylene;
ring

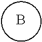

represents substituted or unsubstituted arylene, heteroarylene, heterocycloalkylene, heterocycloalkenylene, cycloalkylene, or cycloalkenylene;
R¹ and R² each independently represent H, alkyl, or cycloalkyl;
X represents a bond, O, S, or NH; and
R³ represents H, alkyl, cycloalkyl, (cycloalkyl)alkyl, heterocycloalkyl, (heterocycloalkyl)alkyl, aryl, arylalkyl, heteroaryl, or heteroarylalkyl.

In some embodiments, ring

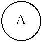

represents substituted or unsubstituted arylene, such as substituted or unsubstituted phenylene.

In certain embodiments, the compound has the structure of formula Ia:

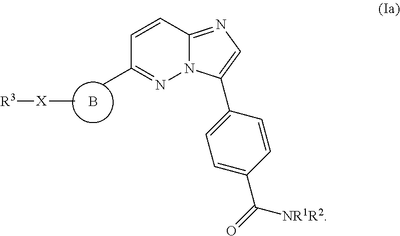

In some embodiments, ring

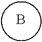

represents substituted or unsubstituted arylene, such as substituted or unsubstituted phenylene.

In certain embodiments, the compound has the structure of formula Ib:

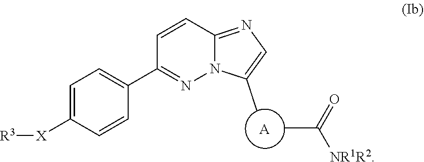

In some embodiments, ring

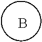

represents substituted or unsubstituted heterocycloalkylene, such as substituted or unsubstituted N-containing heterocycloalkylene.

In certain such embodiments, X is O and R³ is (heterocycloalkyl)alkyl.

In certain embodiments, the compound has the structure of formula Ic:

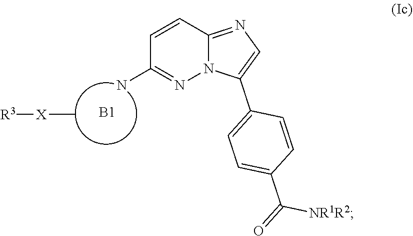

wherein ring

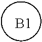

is a substituted or unsubstituted N-containing heterocycloalkylene.

In certain embodiments, the compound has the structure of formula Id:

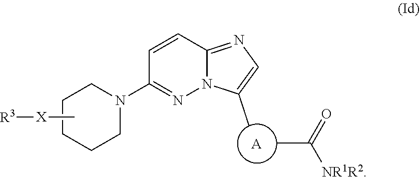

In certain embodiments, the compound has the structure of formula Ie:

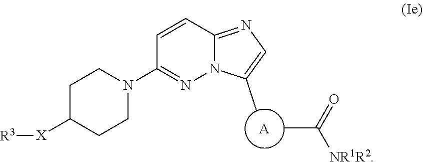

Alternatively, in some embodiments, the compound has the structure of formula If:

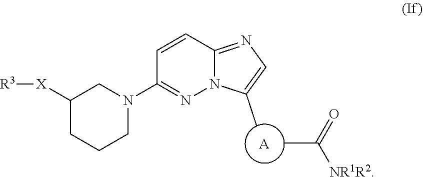

In certain such embodiments (i.e., a compound of formula Id, Ie, If), X—R³ is NH₂.

In still further alternative embodiments, the compound has the structure of formula Ig:

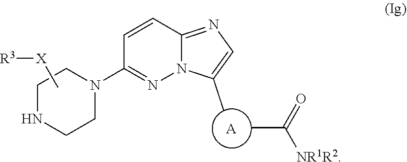

In any of the foregoing embodiments, X may be a bond. Alternatively, X may be O. In still further embodiments, X may be NH.

In certain embodiments, R³ is H. Alternatively, R³ may be alkyl. Alternatively still, R³ may be (heterocycloalkyl)alkyl.

In some embodiments, X—R³ is H. Alternatively, in some embodiments, X—R³ is not

H.

In certain embodiments, R¹ and R² are each independently H or alkyl, preferably H.

In certain embodiments, the compound of the invention is selected from

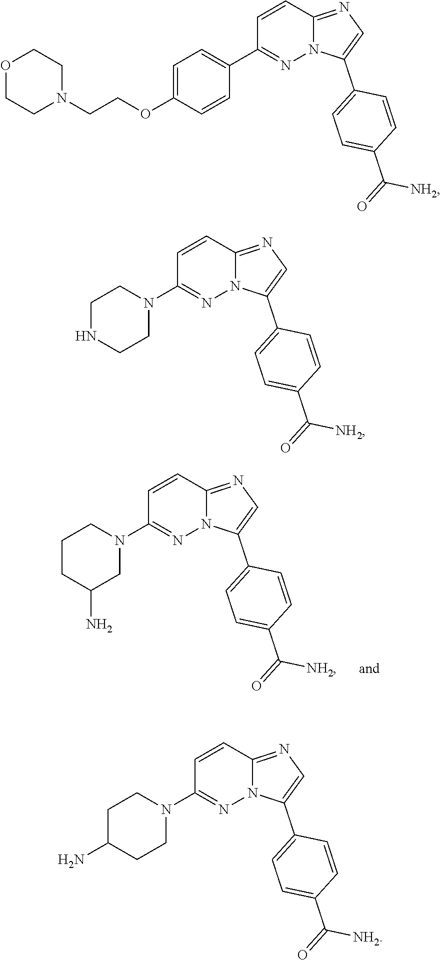 [SMILES: NC(=O)c1ccc(-c2cnc3ccc(-c4ccc(OCCN5CCOCC5)cc4)nn23)cc1 ; NC(=O)c1ccc(-c2cnc3ccc(N4CCNCC4)nn23)cc1 ; NC(=O)c1ccc(-c2cnc3ccc(N4CCCC(N)C4)nn23)cc1 ; "and" ; NC(=O)c1ccc(-c2cnc3ccc(N4CCC(N)CC4)nn23)cc1]

In certain embodiments, the invention provides a compound of formula II,

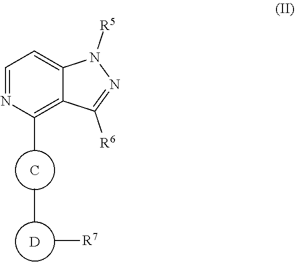

or a pharmaceutically acceptable salt thereof;
wherein:
ring

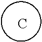

represents substituted or unsubstituted arylene or heteroarylene;
ring

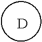

represents substituted or unsubstituted heterocycloalkylene or heterocycloalkenylene;
R⁵ represents H or alkyl;
R⁶ represents H, alkyl, alkoxy, or amino; and
R⁷ represents substituted or unsubstituted aryl or heteroaryl.

In some embodiments, ring

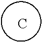

represents substituted or unsubstituted arylene, such as substituted or unsubstituted phenylene.

In some embodiments, the compound has the structure of formula IIb:

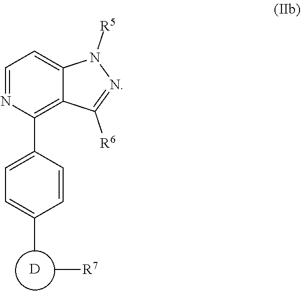

In certain embodiments, ring

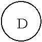

represents substituted or unsubstituted heterocycloalkylene, such as substituted or unsubstituted N-containing heterocycloalkylene.

In some embodiments, the compound has the structure of formula IIc:

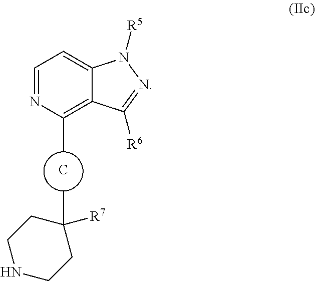

In any of the foregoing embodiments, R⁷ may be substituted or unsubstituted aryl, such as substituted or unsubstituted phenyl. For example, R⁷ may be phenyl substituted by halo (e.g., chloro).

In certain embodiments, R⁵ represents H.

In certain embodiments, R⁶ represents alkyl.

In certain embodiments, the compound is

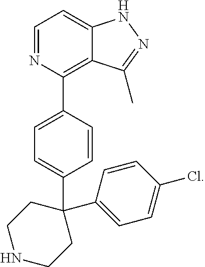 [SMILES: Cc1n[nH]c2ccnc(-c3ccc(C4(c5ccc(Cl)cc5)CCNCC4)cc3)c12]

In certain embodiments, the compound of the invention is:

4-(6-(4-(2-morpholinoethoxy)phenyl)imidazo[1,2-b]pyridazin-3-yl)benzamide;
4-(6-(piperazin-1-yl)imidazo[1,2-b]pyridazin-3-yl)benzamide;
4-(6-(3 -aminopiperidin-1-yl)imidazo[1,2-b]pyridazin-3-yl)benzamide;
4-(6-(4-aminopiperidin-1-yl)imidazo[1,2-b]pyridazin-3-yl)benzamide; or
4-(4-(4-(4-chlorophenyl)piperidin-4-yl)phenyl)-3-methyl-1H-pyrazolo[4,3-c]pyridine;
or a pharmaceutically acceptable salt thereof.

The present invention also encompasses pharmaceutically acceptable salts of the compounds of formula I or II. Exemplary pharmaceutically acceptable salts include, but are not limited to, alkyl, dialkyl, trialkyl or tetra-alkyl ammonium salts; or salts of L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc. In certain embodiments, contemplated salts of the invention include, but are not limited to, Na, Ca, K, Mg, Zn or other metal salts.

In other embodiments, the present invention encompasses an acid addition salt of a compound of formula I or II. In such embodiments, the acid addition salt may be obtained by treating the free base with an acid such as an inorganic acid, for example, hydrochloric acid, hydrobromic acid, sulfuric acid, phosphoric acid, nitric acid and the like; or an organic acid such as for example, acetic acid, hydroxyacetic acid, propanoic acid, lactic acid, pyruvic acid, malonic acid, fumaric acid, maleic acid, oxalic acid, tartaric acid, succinic acid, malic acid, ascorbic acid, benzoic acid, tannic acid, pamoic acid, citric acid, methylsulfonic acid, ethanesulfonic acid, benzenesulfonic acid, formic and the like (Handbook of Pharmaceutical Salts, P. Heinrich Stahal & Camille G. Wermuth (Eds), Verlag Helvetica Chemica Acta-Zürich, 2002, 329-345).

In still further embodiments, the present invention encompasses a base addition salt of a compound of formula I or II. In such embodiments, the base addition salt may be treating the compound with an appropriate base such as an inorganic base, for example, sodium hydroxide, magnesium hydroxide, potassium hydroxide, calcium hydroxide, ammonia and the like; or an organic base such as for example, L-arginine, ethanolamine, betaine, benzathine, morpholine and the like. (Handbook of Pharmaceutical Salts, P. Heinrich Stahal & Camille G. Wermuth (Eds), Verlag Helvetica Chemica Acta-Zürich, 2002, 329-345).

The present invention also encompasses pharmaceutically acceptable solvates of the compounds of formula I or II, such as solvates with methanol, ethanol, dimethylformamide, and the like. Mixtures of such solvates can also be prepared. Also encompassed are hydrates of the compounds of the invention (i.e., wherein the compound exists as a solvate with water). The source of such solvate can be from the solvent of crystallization, inherent in the solvent of preparation or crystallization, or adventitious to such solvent.

### Pharmaceutical Compositions

The present invention also provides pharmaceutical compositions comprising a compound of formula (I) or (II), and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively, or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatable with such fluids. A preferred route of administration is local administration (e.g., topical administration, such as eye drops, or administration via an implant).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans, and other mammals such as equines, cattle, swine and sheep; and poultry and pets in general.

In certain embodiments, compounds of the invention may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

This invention includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Methods of Use

In some embodiments, the invention provides methods of treating cancer, comprising administering to a subject in need thereof a compound of the invention, or a pharmaceutical composition comprising the same.

Cancers treatable by the methods of the invention include acute lymphoblastic leukemia (ALL), acute myeloid leukemia (AML), adrenocortical carcinoma, Kaposi sarcoma, anal cancer, brain cancer, bile duct cancer, bladder cancer, breast cancer, bronchial tumor, Burkitt lymphoma, cervical cancer, chronic lymphocytic leukemia (CLL), chronic myelogenous leukemia (CML), chronic myeloproliferative neoplasms, colorectal cancer, cutaneous T-cell lymphoma, esophageal cancer, Ewing Sarcoma, extragonadal germ cell tumor, eye cancer, fallopian tube cancer, gallbladder cancer, gastric (stomach) cancer, gastrointestinal carcinoid tumor, gastrointestinal stromal tumors (GIST), Hairy cell leukemia, head and neck cancer, Hodgkin Lymphoma, islet cell tumors, pancreatic neuroendocrine tumors, Kaposi Sarcoma, renal cell cancer, Langerhans Cell histiocytosis, leukemia, liver cancer, lung cancer (non-small cell and small cell), lymphoma, malignant fibrous histiocytoma of bone, melanoma, mesothelioma, multiple myeloma, myelodysplastic/myeloproliferative neoplasms, neuroblastoma, Non-Hodgkin lymphoma, non-small cell lung cancer, oral cancer, osteosarcoma, ovarian cancer, pancreatic cancer, pancreatic ductal adenocarcinoma, paraganglioma, parathyroid cancer, penile cancer, pheochromocytoma, pituitary tumor, primary Central Nervous System (CNS) lymphoma, primary peritoneal cancer, prostate cancer, rectal cancer, skin cancer, small intestine cancer, testicular cancer, thymoma and thymic carcinoma, thyroid cancer, urethral cancer, uterine cancer, vaginal cancer, vascular tumors, vulvar cancer, and Wilms Tumor.

As detailed above, the LATS1/2 inhibitors of the invention sensitize cancer cells to anticancer cytotoxic drugs (Gujral, Taran; Kirschner, Marc, 2016, PNAS). Accordingly, in some embodiments, the method of treating cancer comprises conjointly administering one or more chemotherapeutic agents.

Exemplary chemotherapeutic agents useful in the methods of the invention includes aminoglutethimide, amsacrine, anastrozole, asparaginase, Bacillus Calmette-Guérin vaccine (bcg), bicalutamide, bleomycin, buserelin, busulfan, campothecin, capecitabine, carboplatin, carmustine, chlorambucil, chloroquine, cisplatin, cladribine, clodronate, colchicine, cyclophosphamide, cyproterone, cytarabine, dacarbazine, dactinomycin, daunorubicin, demethoxyviridin, dichloroacetate, dienestrol, diethylstilbestrol, docetaxel, doxorubicin, epirubicin, estradiol, estramustine, etoposide, everolimus, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, letrozole, leucovorin, leuprolide, levamisole, lomustine, lonidamine, mechlorethamine, medroxyprogesterone, megestrol, melphalan, mercaptopurine, mesna, metformin, methotrexate, mitomycin, mitotane, mitoxantrone, nilutamide, nocodazole, octreotide, oxaliplatin, paclitaxel, pamidronate, pentostatin, perifosine, plicamycin, porfimer, procarbazine, raltitrexed, rituximab, sorafenib, streptozocin, sunitinib, suramin, tamoxifen, temozolomide, temsirolimus, teniposide, testosterone, thioguanine, thiotepa, titanocene dichloride, topotecan, trastuzumab, tretinoin, vinblastine, vincristine, vindesine, or vinorelbine.

### Methods of Synthesis

The present invention also provides methods of synthesizing the compounds of Formula I and II. Compounds of the invention may be prepared according to conventional methods known to persons skilled in the art of synthetic organic chemistry. Provided below is an exemplary route to the compounds of Formula I:

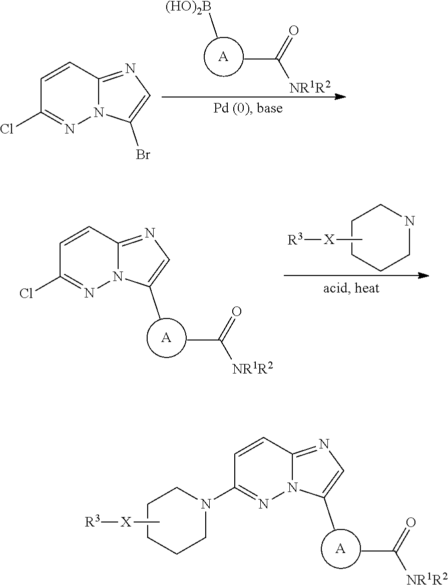

In step 1, a cross-coupling reaction such as a Pd(0)-catalyzed Suzuki cross-coupling reaction may be used to form the intermediate. Suitable Pd(0) catalysts include Pd(Ph₃P)₄ and Pd₂(dba)₃. Analogous Ni(0) and Pt(0) catalysts may also be used, as may Pd(II) catalysts used with a reductant. Suitable bases include inorganic bases such as Cs₂CO₃. In step 2, acid may optionally be used to remove the protecting group to yield the final product. Suitable acids include, for example, HCl and trifluoroacetic acid.

### EXEMPLIFICATION

### General Information

Characterization of the compounds was performed using NMR spectroscopy, recorded on 300 and/or 600 MHz Varian and acquired at room temperature. Chemical shifts are given in ppm referenced either to internal TMS or to the solvent signal.

Compounds of the invention were generally purified by column chromatography (Auto-column) on a Teledyne-ISCO CombiFlash with a silica column, unless noted otherwise.

The following abbreviations are used in the examples:

nBu₄NOH tetrabutylammonium hydroxide

DMF dimethylformamide

MPLC medium-pressure liquid chromatography

MeOH methanol

NaCNBH₃ Sodium cyanoborohydride

NaOMe sodium methoxide

EtOH ethanol

CDCl₃ deuterated chloroform

NaBH₄ sodium borohydride

MgSO₄ magnesium sulfate

HC1 hydrochloric acid

Et₂O ether

NH₄Cl ammonium chloride

DIBAL-H diisobutylaluminum hydride

K₂CO₃ potassium carbonate

CH₂Cl₂ dichloromethane

CuI copper iodide

NMO N-Methylmorpholine oxide

SiO₂ silica gel

### EXAMPLE 1

### Synthesis of 4-(6-(4-(2-Morpholinoethoxy)phenyl)imidazo[1,2-b]pyridazin-3-yl)benzamide (Compound 1)

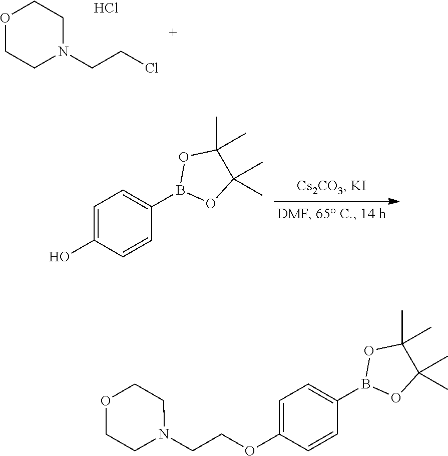

4-(2-(4-(4,4,5,5-Tetramethyl-1,3,2-dioxaborolan-2-yl)phenoxy)ethyl)morpholine: 4-(4,4,5,5-Tetramethyl-1,3,2-dioxaborolan-2-yl)phenol (250 mg, 1.14 mmol), 4-(2-chloroethyl)morpholine hydrochloride (233 mg, 1.25 mmol), cesium carbonate (815 mg, 2.50 mmol) and potassium iodide (20 mg, 0.12 mmol) were mixed in DMF (10 mL) and stirred at 65° C. for 14 hours, then cooled to room temperature. The reaction mixture was diluted with EtOAc and then washed with water, dried (MgSO₄) and concentrated. Column chromatography (3/1 EtOAc/hex) followed by concentration with rotary evaporator gave the desired title compound as a light brown solid.

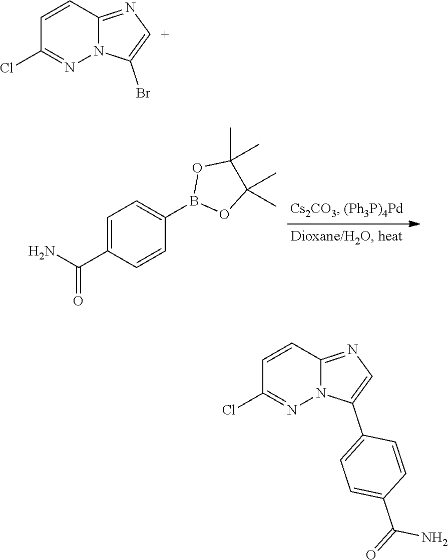

4-(6-Chloroimidazo[1,2-b]pyridazin-3-yl)benzamide: 3-Bromo-6-chloroimidazo[1,2-b]pyridazine (500 mg, 2.15 mmol), 4-(4,4,5,5-tetramethyl-1,3,2-dioxaborolan-2-yl)benzamide (531 mg, 2.15 mmol), cesium carbonate (841 mg, 2.58 mmol) and tetrakis(triphenylphosphine)palladium(0) (62 mg, 0.05 mmol) were mixed in dioxane and water (7.0 mL/1.5 mL) and stirred at 80° C. for 2 hours then 70° C. for 4 hours, after that cooled to room temperature. The reaction mixture was diluted with EtOAc and then washed with water, dried (MgSO₄) and concentrated. Column chromatography (3/1 EtOAc/hex) followed by concentration with rotary evaporator gave the desired title compound as a yellow solid.

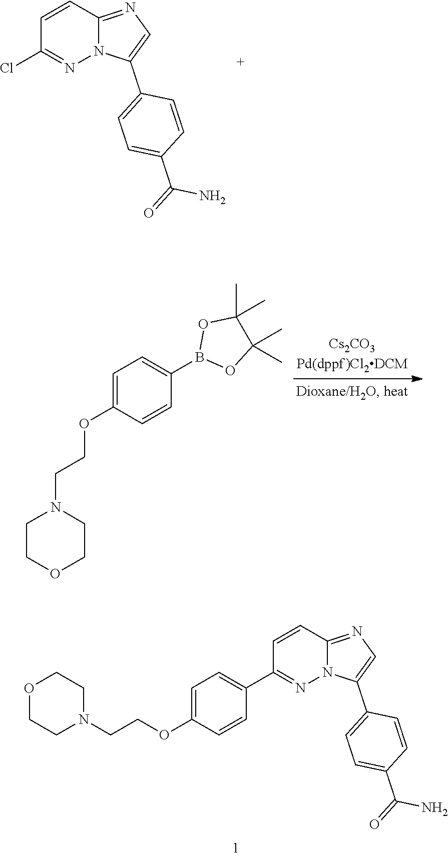 [SMILES: NC(=O)c1ccc(-c2cnc3ccc(Cl)nn23)cc1 ; "+" ; CC1(C)OB(c2ccc(OCCN3CCOCC3)cc2)OC1(C)C ; NC(=O)c1ccc(-c2cnc3ccc(-c4ccc(OCCN5CCOCC5)cc4)nn23)cc1]

4-(6-(4-(2-Morpholinoethoxy)phenyl)imidazo[1,2-b]pyridazin-3-yl)benzamide (1): 4-(6-Chloroimidazo[1,2-b]pyridazin-3- yl)benzamide (50 mg, 0.18 mmol), 4-(2-(4-(4,4,5,5-tetramethyl-1,3,2-dioxaborolan-2-yl)phenoxy)ethyl)morpholine (67 mg, 0.20 mmol), cesium carbonate (72 mg, 0.22 mmol) and Pd(dppf)Cl₂.DCM (7.5 mg, 0.009 mmol) were mixed in dioxane and water (1.0 mL/0.5 mL) and stirred at 120° C. for 75 minutes, after that cooled to room temperature. The reaction mixture was diluted with EtOAc and then washed with water, dried (MgSO₄) and concentrated. Chromatography (3/1 EtOAc/hex) then concentration with rotatory evaporator gave the desired title compound (compound 1) as a solid. Spectroscopic data: ¹H NMR (400 MHz, DMSO-d₆) δ ppm 8.38-8.35 (m, 3 H), 8.27 (d, J=9.6 Hz, 1 H), 8.19 (s 1 H), 8.12-8.05 (m, 4 H), 7.9 (d, J=9.6 Hz, 1 H), 7.41 (br s, 1 H), 7.15 (d, J=8.9 Hz, 1 H), 4.20 (t, J=5.7 Hz, 2 H), 3.6 (t, J=4.6 Hz, 4 H), 2.74 (t, J=5.7 Hz, 2 H), 2.51-2.48 (m, 4 H).

### EXAMPLE 2

### Synthesis of 4-(6-(piperazin-1-yl)imidazo[1,2-b]pyridazin-3-yl)benzamide (Compound 2)

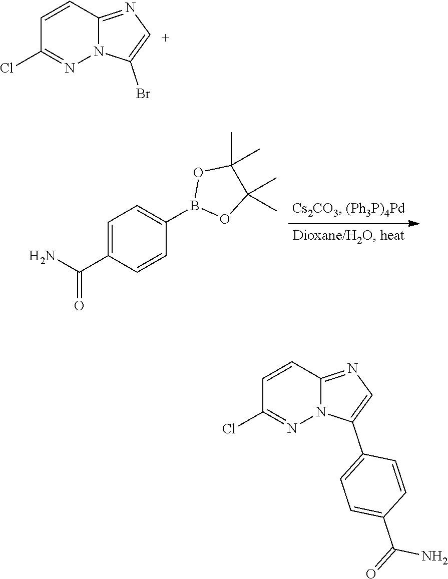

4-(6-Chloroimidazo[1,2-b]pyridazin-3-yl)benzamide: 3-Bromo-6-chloroimidazo[1,2-b]pyridazine (500 mg, 2.15 mmol), 4-(4,4,5,5-tetramethyl-1,3,2-dioxaborolan-2-yl)benzamide (531 mg, 2.15 mmol), cesium carbonate (841 mg, 2.58 mmol) and tetrakis(triphenylphosphine)palladium(0) (62 mg, 0.05 mmol) were mixed in dioxane and water (7.0 mL/1.5 mL) and stirred at 80° C. for 2 hours then 70° C. for 4 hours, after that cooled to room temperature. The reaction mixture was diluted with EtOAc and then washed with water, dried (MgSO₄) and concentrated. Chromatography (3/1 EtOAc/hex) then concentration with rotatory evaporator gave the desired title compound as a yellow solid.

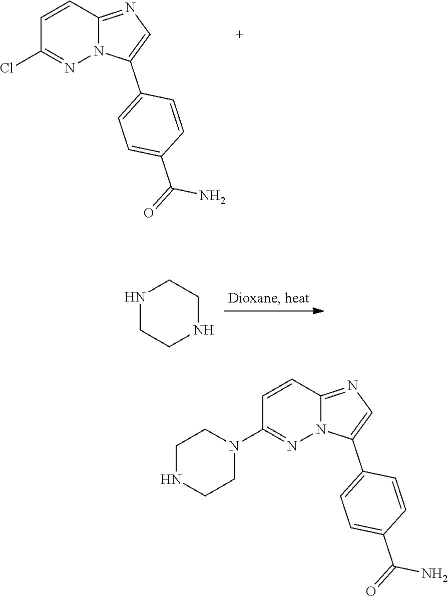 [SMILES: NC(=O)c1ccc(-c2cnc3ccc(Cl)nn23)cc1 ; "+" ; C1CNCCN1 ; NC(=O)c1ccc(-c2cnc3ccc(N4CCNCC4)nn23)cc1]

4-(6-(Piperazin-1-yl)imidazo[1,2-b]pyridazin-3-yl)benzamide (2): 4-(6-Chloroimidazo[1,2-b]pyridazin-3-yl)benzamide (50 mg, 0.18 mmol) and piperazine (200 mg, 2.32 mmol) were mixed in dioxane (0.5 mL) and stirred at 120° C. for 75 minutes, after that cooled to room temperature. The reaction mixture was diluted with EtOAc and then washed with water, dried (MgSO₄) and concentrated. Chromatography (3/1 EtOAc/hex) then concentration with rotatory evaporator gave the desired title compound as a solid. Spectroscopic data: ¹H NMR (400 MHz, DMSO-d₆) δ ppm 8.28-8.25 (m, 3 H), 8.11 (s, 1 H), 8.01-7.96 (m, 3 H), 7.93 (d, J=10.0 Hz, 1 H), 7.35 (br s, 1 H), 7.25 (d, J=10.0 Hz, 1 H), 3.48-3.44 (m, 4 H), 2.85 (t, J=5.1 Hz, 4 H).

### EXAMPLE 3

### Synthesis of 4-(6-(3-Aminopiperidin-1-yl)imidazo[1,2-b]pyridazin-3-yl)benzamide (Compound 3)

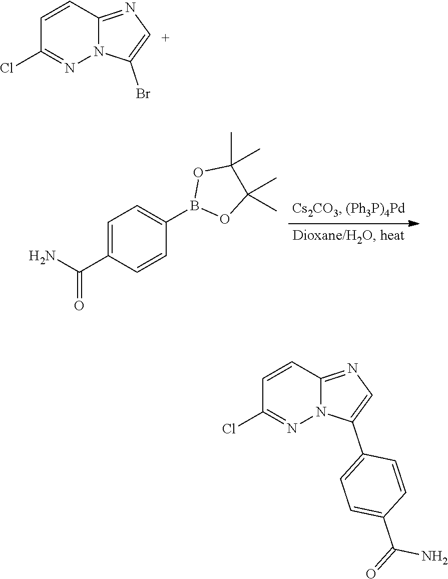

4-(6-Chloroimidazo[1,2-b]pyridazin-3-yl)benzamide: 3-Bromo-6-chloroimidazo[1,2-b]pyridazine (500 mg, 2.15 mmol), 4-(4,4,5,5-tetramethyl-1,3,2-dioxaborolan-2-yl)benzamide (531 mg, 2.15 mmol), cesium carbonate (841 mg, 2.58 mmol) and tetrakis(triphenylphosphine)palladium(0) (62 mg, 0.05 mmol) were mixed in dioxane and water (7.0 mL/1.5 mL) and stirred at 80° C. for 2 hours then 70° C. for 4 hours, after that cooled to room temperature. The reaction mixture was diluted with EtOAc and then washed with water, dried (MgSO₄) and concentrated. Chromatography (3/1 EtOAc/hex) then concentration with rotary evaporator gave the desired title compound as a yellow solid.

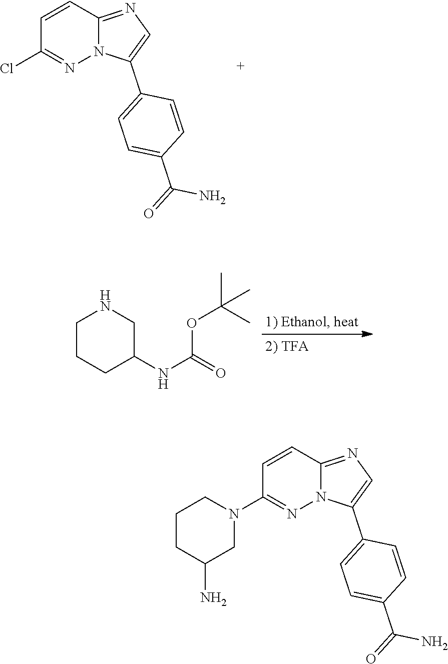 [SMILES: NC(=O)c1ccc(-c2cnc3ccc(Cl)nn23)cc1 ; "+" ; CC(C)(C)OC(=O)NC1CCCNC1 ; NC(=O)c1ccc(-c2cnc3ccc(N4CCCC(N)C4)nn23)cc1]

4-(6-(3-Aminopiperidin-1-yl)imidazo[1,2-b]pyridazin-3-yl)benzamide (3): 4-(6-Chloroimidazo[1,2-b]pyridazin-3- yl)benzamide (75 mg, 0.28 mmol) and 3-N-Boc-amino-piperazine (470 mg, 2.35 mmol) were mixed in ethanol (0.7 mL) in a sealed tube and heated at 145° C. for 2 hours, after that cooled to room temperature. The reaction mixture was diluted with EtOAc and then washed with water, dried (MgSO₄) and concentrated. Chromatography (3/1 EtOAc/hex) then concentration with rotatory evaporator gave the desired intermediate Boc-protected 4-(6-(3-aminopiperidin-1-yl)imidazo[1,2-b]pyridazin-3-yl)benzamide. This intermediate (100 mg) was then mixed with TFA (745 mg, 6.53 mmol) in DCM (1.o mL) at 0° C. then warmed to room temperature and stirred for 2 hours and concentrated.
Chromatography (EtOAc) then concentration with rotatory evaporator gave the desired title compound (compound 3). Spectroscopic data: ¹H NMR (400 MHz, DMSO-d₆) δ ppm 8.39 (s, 1 H), 8.27 (d, J=8.6 Hz, 2 H), 8.11 (s, 1 H), 8.05-7.98 (m, 2 H), 7.94 (d, J=10.0 Hz, 1 H), 7.35 (br s, 1 H), 7.25 (d, J=10.0 Hz, 1 H), 3.69-3.27 (m, 9 H).

### EXAMPLE 4

### Synthesis of 4-(6-(4-Aminopiperidin-1-yl)imidazo[1,2-b]pyridazin-3-yl)benzamide (compound 4)

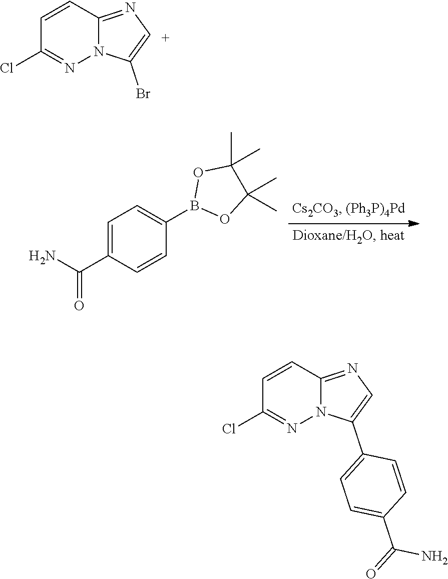

4-(6-Chloroimidazo[1,2-b]pyridazin-3-yl)benzamide: 3-Bromo-6-chloroimidazo[1,2-b]pyridazine (500 mg, 2.15 mmol), 4-(4,4,5,5-tetramethyl-1,3,2-dioxaborolan-2-yl)benzamide (531 mg, 2.15 mmol), cesium carbonate (841 mg, 2.58 mmol) and tetrakis(triphenylphosphine)palladium(0) (62 mg, 0.05 mmol) were mixed in dioxane and water (7.0 mL/1.5 mL) and stirred at 80° C. for 2 hours then 70° C. for 4 hours, after that cooled to room temperature. The reaction mixture was diluted with EtOAc and then washed with water, dried (MgSO₄) and concentrated. Chromatography (3/1 EtOAc/hex) then concentration with rotatory evaporator gave the desired title compound as a yellow solid.

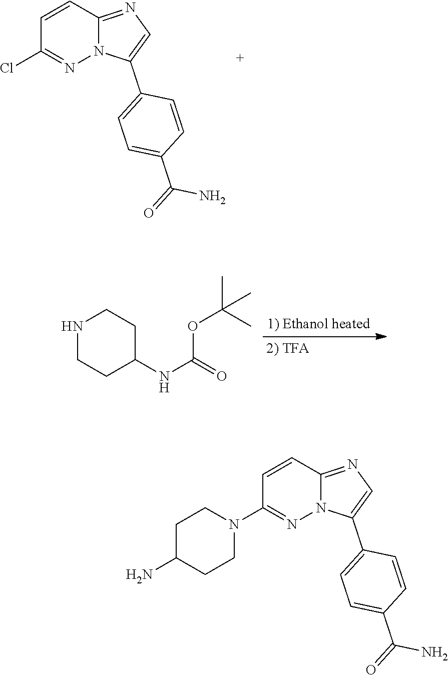 [SMILES: NC(=O)c1ccc(-c2cnc3ccc(Cl)nn23)cc1 ; "+" ; CC(C)(C)OC(=O)NC1CCNCC1 ; NC(=O)c1ccc(-c2cnc3ccc(N4CCC(N)CC4)nn23)cc1]

4-(6-(4-Aminopiperidin-1-yl)imidazo[1,2-b]pyridazin-3-yl)benzamide (4): 4-(6-Chloroimidazo[1,2-b]pyridazin-3- yl)benzamide (75 mg, 0.28 mmol) and 4-N-Boc-amino-piperazine (470 mg, 2.35 mmol) were mixed in ethanol (0.7 mL) in a sealed tube and heated at 145° C. for 2 hours, after that cooled to room temperature. The reaction mixture was diluted with EtOAc and then washed with water, dried (MgSO₄) and concentrated. Chromatography (3/1 EtOAc/hex) then concentration with rotatory evaporator gave the desired intermediate Boc-protected 4-(6-(4-aminopiperidin-1-yl)imidazo[1,2-b]pyridazin-3-yl)benzamide. This intermediate (100 mg) was then mixed with TFA (745 mg, 6.53 mmol) in DCM (1.0 mL) at 0° C. then warmed to room temperature and stirred for 2 hours and concentrated.
Chromatography (EtOAc) then concentration with rotary evaporator gave the desired title compound (compound 4). Spectroscopic data: ¹H NMR (400 MHz, DMSO-d₆) δ ppm 8.40 (s, 1 H), 8.29-8.23 (m, 2 H), 8.12 (m, 1 H), 8.04-7.93 (m, 3 H), 7.37 (br s, 1 H), 7.29 (d, J=10.0 Hz, 1 H), 3.74-3.19 (m, 9 H).

### EXAMPLE 5

### Synthesis of 4-(4-(4-(4-chlorophenyl)piperidin-4-yl)phenyl)-3-methyl-1H-pyrazolo[4,3-c]pyridine (Compound 5)

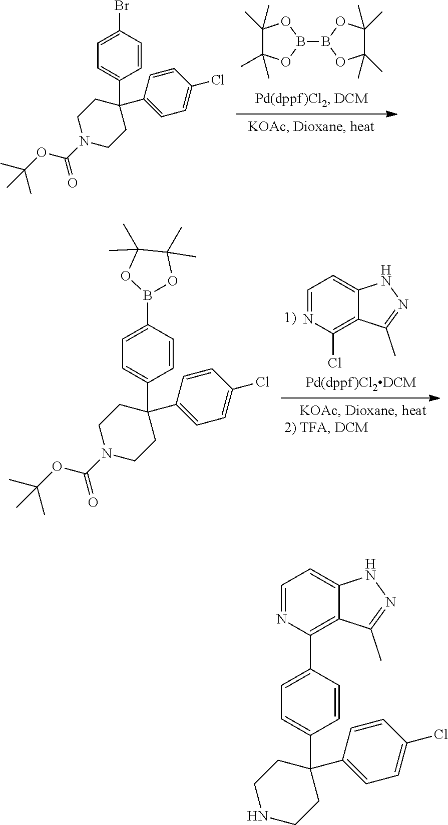

tert-Butyl 4-(4-Chlorophenyl)-4-(4-(4,4,5,5-tetramethyl-1,3,2-dioxaborolan-2-yl)phenyl)piperidine-1-carboxylate: tert-butyl 4-(4-bromophenyl)-4-(4-chlorophenyl)piperidine-1-carboxylate (200 mg, 0.44 mmol), 4,4,4′,4′,5,5,5′,5′-octamethyl-2,2′-bi(1,3,2-dioxaborolane) (134 mg, 0.52 mmol), potassium acetate (130 mg, 1.32 mmol) and Pd(dppf)Cl₂.DCM (36 mg, 0.04 mmol) were mixed in dioxane (7.0 mL) and stirred at 110 ° C. for 1.5 hours, after that cooled to room temperature. The reaction mixture was diluted with EtOAc and then washed with water, dried (MgSO₄) and concentrated. Chromatography (3/1 EtOAc/hex) then concentration with rotatory evaporator gave the desired title compound as a solid.
4-(4-(4-(4-Chlorophenyl)piperidin-4-yl)phenyl)-3-methyl-1H-pyrazolo[4,3-c]pyridine (5): tert-Butyl 4-(4-Chlorophenyl)-4-(4-(4,4,5,5-tetramethyl-1,3,2-dioxaborolan-2-yl)phenyl)piperidine-1-carboxylate (obtained from the previous step, 176 mg, 0.35 mmol), 4-chloro-3-methyl-1H-pyrazolo[4,3-c]pyridine (65 mg, 0.39 mmol), sodium carbonate (1.5 mg, 1.23 mmol) and Pd(dppf)Cl₂.DCM (14 mg, 0.02 mmol) were mixed in dioxane (4.5 mL) and stirred at 110° C. for 30 minutes, after that cooled to room temperature. The reaction mixture was diluted with EtOAc and then washed with water, dried (MgSO₄) and concentrated. Chromatography (3/1 EtOAc/hex) then concentration with rotatory evaporator gave the desired intermediate Boc-protected 4-(4-(4-(4-Chlorophenyl)piperidin-4-yl)phenyl)-3-methyl-1H-pyrazolo[4,3-c]pyridine. This intermediate (90 mg) was then mixed with TFA (745 mg, 6.53 mmol) in DCM (2.0 mL) at 0° C. then warmed to room temperature and stirred for 2 hours and concentrated. Chromatography (EtOAc) then concentration with rotatory evaporator gave the desired title compound (compound 5). Spectroscopic data: ¹H NMR (400 MHz, DMSO-d₆) δ ppm 8.30 (d, J=5.9 Hz, 2 H), 7.58 (d, J=8.5 Hz, 2 H), 7.48 (d, J=8.5 Hz, 2 H), 8.45-7.38 (m, 4 H), 2.94 (t, J=5.24 Hz, 4 H), 2.52-2.47 (m, 4 H), 2.20 (s, 3 H).

### EXAMPLE 6

### Biochemical & Biological Data

IC₅₀ scores were determined using a TR-FRET LanthaScreen binding assay using LATS1 (amino acids 589-1130; ThermoFisher, PV6363), LATS2 (amino acids 488-1080; ThermoFisher, PV6366); kinase tracer 236 (ThermoFisher, PV5592); LanthaScreen Eu-anti-GST (ThermoFisher, PV3189); 5× kinase buffer A (ThermoFisher, PV6135); low-volume 384-well plate format. Activity potency results are shown in the Table below.

| | LATS1/2 |
|---|---|
| | IC₅₀ |
| IUPAC Name | (nM) |
| 4-(6-(4-(2-morpholinoethoxy)phenyl)imidazo[1,2-b] | ++ |
| pyridazin-3-yl)benzamide; Compound 1 | |
| 4-(6-(piperazin-1-yl)imidazo[1,2-b]pyridazin-3-yl) | +++ |
| benzamide | |
| 4-(6-(3-aminopiperidin-1-yl)imidazo[1,2-b]pyridazin-3-yl) | +++ |
| benzamide; Compound 3 | |
| 4-(6-(4-aminopiperidin-1-yl)imidazo[1,2-b]pyridazin-3-yl) | +++ |
| benzamide; Compound 4 | |
| 4-(4-(4-(4-chlorophenyl)piperidin-4-yl)phenyl)-3-methyl- | +++ |
| 1H-pyrazolo[4,3-c]pyridine; Compound 5 | |

+ represents potency between 1 μM and 5 μM, ++ represents potency from 0.01 μM to 1.0 μM, +++ represents potency from 0.0001 to 0.05 μM (IC₅₀).

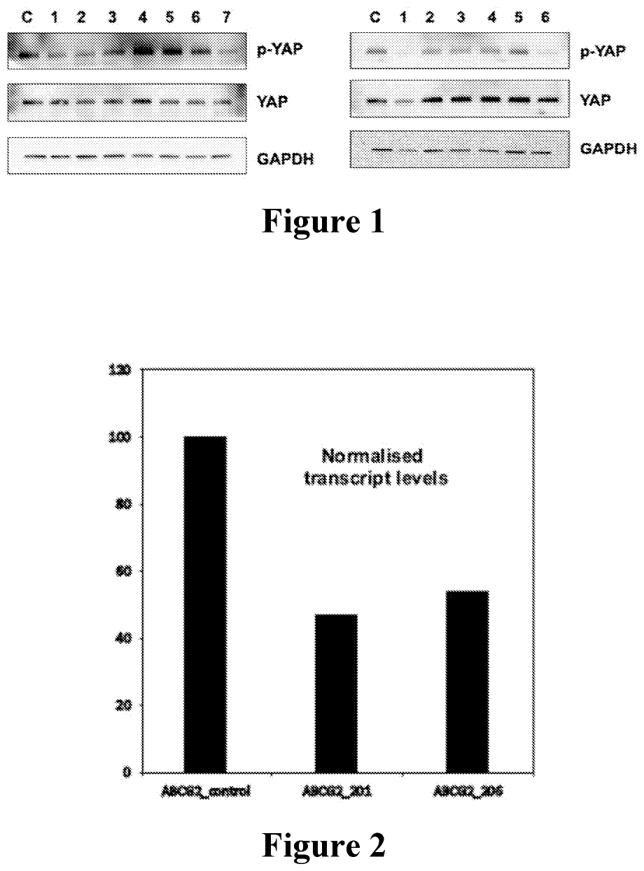

The LATS1/2 inhibitors of the invention demonstrate the ability to reduce YAP phosphorylation in pancreatic ductal adenocarcinoma (PDAC) PANC1 cells in a dose-dependent manner at 48 h. Results are shown in FIG. 1.

As shown in FIG. 1, Compounds 1 and 2 particularly reduce p-YAP levels without impacting overall YAP levels; Compound 5 also reduced p-YAP levels at the lower dose but killed all PANC1 cells at the higher dose. YAP is stained for Yes-associated protein; p-YAP is stained for phosphorylated Yes-associated protein; GAPDH is a standard protein loading control.

As shown in FIG. 2, Compounds 1 and 2 reduce ABCG2 transcription levels when used at 25 μM over a 48-hour treatment period.

### EQUIVALENTS

Having now fully described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

What is claimed is:

1. A compound of formula I, 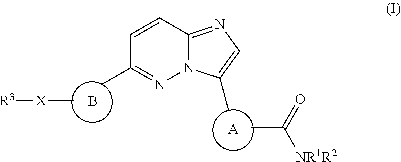
or a pharmaceutically acceptable salt thereof;
wherein:
ring 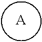
represents substituted or unsubstituted arylene or heteroarylene;
ring 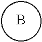
represents substituted or unsubstituted arylene, heteroarylene, heterocycloalkylene, heterocycloalkenylene, cycloalkylene, or cycloalkenylene;
R¹ and R² each independently represent H, alkyl, or cycloalkyl;
X represents a bond, O, S, or NH; and
R³ represents H, alkyl, cycloalkyl, (cycloalkyl)alkyl, heterocycloalkyl, (heterocycloalkyl)alkyl, aryl, arylalkyl, heteroaryl, or heteroarylalkyl.

2. The compound of claim 1, wherein ring 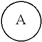
represents substituted or unsubstituted arylene.

3. The compound of claim 1, wherein ring 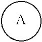
represents substituted or unsubstituted phenylene.

4. The compound of any one of claims 1-3, having the structure of formula Ia:

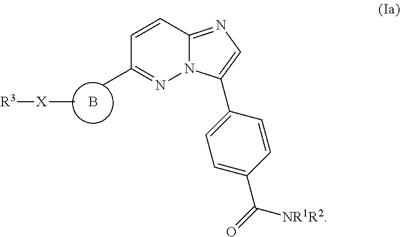

5. The compound of any one of claims 1-4, wherein ring 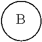
represents substituted or unsubstituted arylene.

6. The compound of any one of claims 1-5, wherein ring 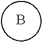
represents substituted or unsubstituted phenylene.

7. The compound of any one of claims 1-6, having the structure of formula Ib:

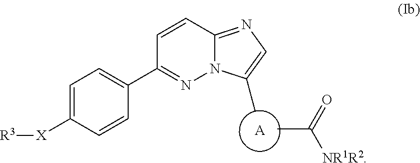

8. The compound of any one of claims 1-4, wherein ring 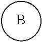
represents substituted or unsubstituted heterocycloalkylene.

9. The compound of any one of claims 1-4 and 8, wherein ring 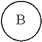
represents substituted or unsubstituted N-containing heterocycloalkylene.

10. The compound of any one of claims 1-4, 8, and 9, having the structure of formula Ic: 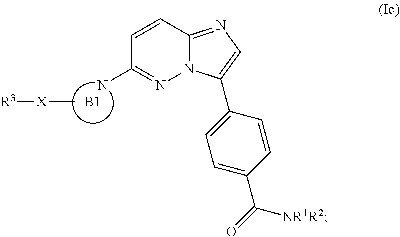
wherein ring 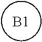
is a substituted or unsubstituted N-containing heterocycloalkylene.

11. The compound of any one of claims 1-4 and 8-10, having the structure of formula Id:

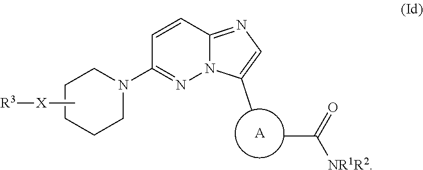

12. The compound of any one of claims 1-4 and 8-11, having the structure of formula Ie:

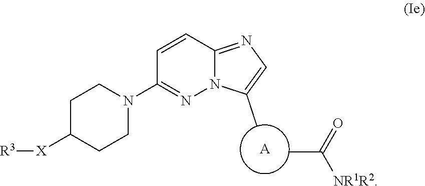

13. The compound of any one of claims 1-4 and 8-11, having the structure of formula If:

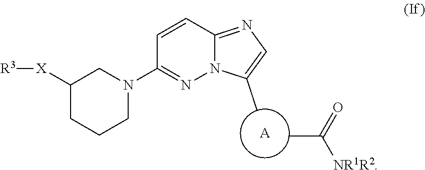

14. The compound of any one of claims 1-4 and 8-10, having the structure of formula Ig:

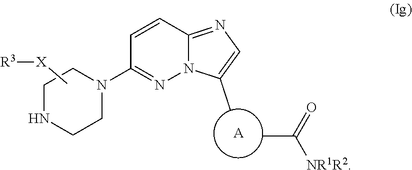

15. The compound of any one of claims 1-14, wherein X is a bond.

16. The compound of any one of claims 1-14, wherein X is O.

17. The compound of any one of claims 1-14, wherein X is NH.

18. The compound of any one of claims 1-17, wherein R³ is H.

19. The compound of any one of claims 1-17, wherein R³ represents alkyl.

20. The compound of any one of claims 1-17, wherein R³ represents (heterocycloalkyl)alkyl.

21. The compound of any one of claims 1-14, wherein X—R³ is H.

22. The compound of any one of claims 1-14, wherein X—R³ is not H.

23. The compound of claim 7, wherein X is O and R³ is (heterocycloalkyl)alkyl.

24. The compound of any one of claims 11-13, wherein X—R³ is NH₂.

25. The compound of any one of claims 1-24, wherein R¹ and R² are each independently H or alkyl.

26. The compound of any one of claims 1-25, wherein R¹ and R² are each H.

27. The compound of claim 1, wherein the compound is selected from:

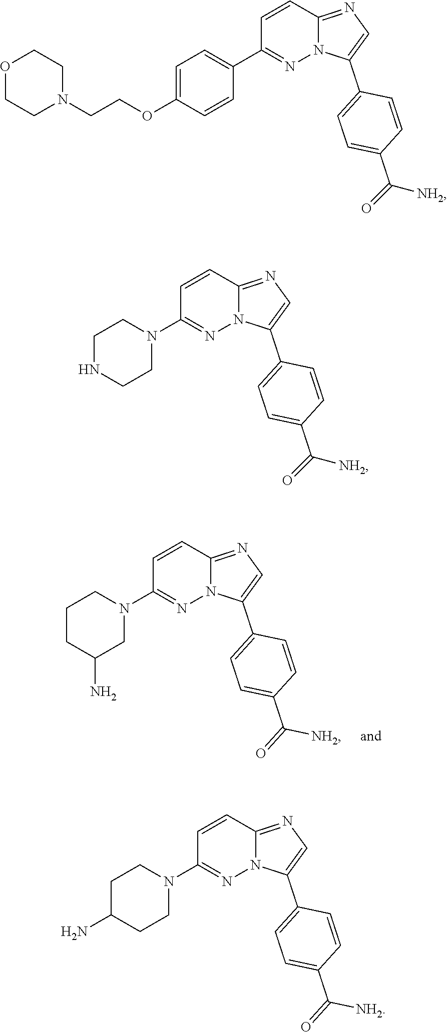 [SMILES: NC(=O)c1ccc(-c2cnc3ccc(-c4ccc(OCCN5CCOCC5)cc4)nn23)cc1 ; NC(=O)c1ccc(-c2cnc3ccc(N4CCNCC4)nn23)cc1 ; NC(=O)c1ccc(-c2cnc3ccc(N4CCCC(N)C4)nn23)cc1 ; "and" ; NC(=O)c1ccc(-c2cnc3ccc(N4CCC(N)CC4)nn23)cc1]

28. A compound of formula II, 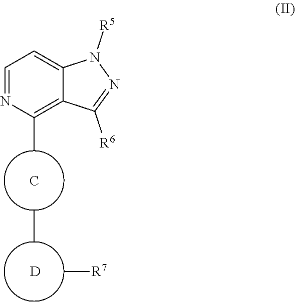
or a pharmaceutically acceptable salt thereof;
wherein:
ring 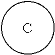
represents substituted or unsubstituted arylene or heteroarylene;
ring 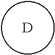
represents substituted or unsubstituted heterocycloalkylene or heterocycloalkenylene;
R⁵ represents H or alkyl;
R⁶ represents H, alkyl, alkoxy, or amino; and
R⁷ represents substituted or unsubstituted aryl or heteroaryl.

29. The compound of claim 28, wherein ring 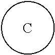
represents substituted or unsubstituted arylene.

30. The compound of claim 28 or 29, wherein ring 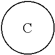
represents substituted or unsubstituted phenylene.

31. The compound of any one of claims 28-30, having the structure of formula IIb:

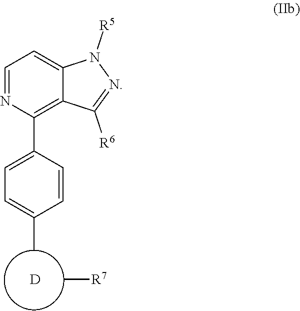

32. The compound of any one of claims 28-31, wherein ring 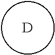
represents substituted or unsubstituted heterocycloalkylene.

33. The compound of any one of claims 28-32, wherein ring 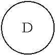
represents substituted or unsubstituted N-containing heterocycloalkylene.

34. The compound of any one of claims 28-33, having the structure of formula IIc:

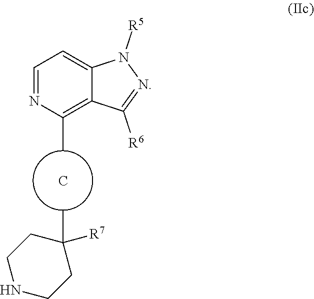

35. The compound of any one of claims 28-34, wherein R⁷ is substituted or unsubstituted aryl.

36. The compound of any one of claims 28-35, wherein R⁷ is substituted or unsubstituted phenyl.

37. The compound of any one of claims 28-36, wherein R⁷ is phenyl substituted by halo (e.g., chloro).

38. The compound of any one of claims 28-37, wherein R⁵ represents H.

39. The compound of any one of claims 28-38, wherein R⁶ represents alkyl.

40. The compound of claim 28, wherein the compound is

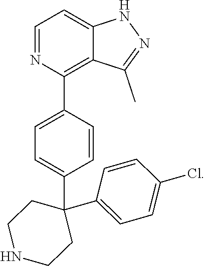 [SMILES: Cc1n[nH]c2ccnc(-c3ccc(C4(c5ccc(Cl)cc5)CCNCC4)cc3)c12]

41. A pharmaceutical composition comprising a compound of any one of claims 1-40, and a pharmaceutically acceptable carrier.

42. A method of treating cancer, comprising administering to a subject in need thereof a compound of any one of claims 1-40 or a pharmaceutical composition of claim 41.

43. The method of claim 42, wherein the cancer is acute lymphoblastic leukemia (ALL), acute myeloid leukemia (AML), adrenocortical carcinoma, Kaposi sarcoma, anal cancer, brain cancer, bile duct cancer, bladder cancer, breast cancer, bronchial tumor, Burkitt lymphoma, cervical cancer, chronic lymphocytic leukemia (CLL), chronic myelogenous leukemia (CML), chronic myeloproliferative neoplasms, colorectal cancer, cutaneous T-cell lymphoma, esophageal cancer, Ewing Sarcoma, extragonadal germ cell tumor, eye cancer, fallopian tube cancer, gallbladder cancer, gastric (stomach) cancer, gastrointestinal carcinoid tumor, gastrointestinal stromal tumors (GIST), Hairy cell leukemia, head and neck cancer, Hodgkin Lymphoma, islet cell tumors, pancreatic neuroendocrine tumors, Kaposi Sarcoma, renal cell cancer, Langerhans Cell histiocytosis, leukemia, liver cancer, lung cancer (non-small cell and small cell), lymphoma, malignant fibrous histiocytoma of bone, melanoma, mesothelioma, multiple myeloma, myelodysplastic/myeloproliferative neoplasms, neuroblastoma, Non-Hodgkin lymphoma, non-small cell lung cancer, oral cancer, osteosarcoma, ovarian cancer, pancreatic cancer, pancreatic ductal adenocarcinoma, paraganglioma, parathyroid cancer, penile cancer, pheochromocytoma, pituitary tumor, primary Central Nervous System (CNS) lymphoma, primary peritoneal cancer, prostate cancer, rectal cancer, skin cancer, small intestine cancer, testicular cancer, thymoma and thymic carcinoma, thyroid cancer, urethral cancer, uterine cancer, vaginal cancer, vascular tumors, vulvar cancer, or Wilms Tumor.

44. The method of claim 42 or 43, wherein the method comprises conjointly administering one or more chemotherapeutic agents.

45. The method of claim 44, wherein the one or more chemotherapeutic agents includes aminoglutethimide, amsacrine, anastrozole, asparaginase, Bacillus Calmette-Guérin vaccine (bcg), bicalutamide, bleomycin, buserelin, busulfan, campothecin, capecitabine, carboplatin, carmustine, chlorambucil, chloroquine, cisplatin, cladribine, clodronate, colchicine, cyclophosphamide, cyproterone, cytarabine, dacarbazine, dactinomycin, daunorubicin, demethoxyviridin, dichloroacetate, dienestrol, diethylstilbestrol, docetaxel, doxorubicin, epirubicin, estradiol, estramustine, etoposide, everolimus, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, letrozole, leucovorin, leuprolide, levamisole, lomustine, lonidamine, mechlorethamine, medroxyprogesterone, megestrol, melphalan, mercaptopurine, mesna, metformin, methotrexate, mitomycin, mitotane, mitoxantrone, nilutamide, nocodazole, octreotide, oxaliplatin, paclitaxel, pamidronate, pentostatin, perifosine, plicamycin, porfimer, procarbazine, raltitrexed, rituximab, sorafenib, streptozocin, sunitinib, suramin, tamoxifen, temozolomide, temsirolimus, teniposide, testosterone, thioguanine, thiotepa, titanocene dichloride, topotecan, trastuzumab, tretinoin, vinblastine, vincristine, vindesine, or vinorelbine.
